(19) United States
(12) Patent Application Publication
(10) Pub. No.: US 2005/0267497 A1
(43) Pub. Date: Dec. 1, 2005

(54) Leak-resistant anastomosis

(76) Inventor: Daniel D. De Oliveira, Lake Jackson, TX (US)

Correspondence Address: OSHA LIANG L.L.P., 1221 MCKINNEY STREET, SUITE 2800, HOUSTON, TX, 77010, US

(21) Appl. No.: 11/128,907

(22) Filed: May 13, 2005

Related U.S. Application Data
(60) Provisional application No. 60/521,522, filed on May 13, 2004.

(51) Int. Cl. A61B 17/08

(52) U.S. Cl. 606/153

(57) ABSTRACT

A graft for a surgical procedure includes a graft body; and a double-layered section attached to the graft body, wherein the double-layered section is configured to sandwich a target to be joined with the graft. A method for implanting a graft includes providing the graft having a double-layered section attached to a graft body; placing a target in the double-layered section such that the double-layered section embraces the target to form a sandwich structure; and joining the graft and the target by securing the sandwich structure.

### CROSS-REFERENCE TO RELATED APPLICATIONS

This claims priority, under 35 U.S.C. § 119, of Provisional Application Ser. No. 60/521,522, filed on May 13, 2004. This Provisional Application is incorporated by reference in its entirety.

### STATEMENT REGARDING FEDERALLY SPONSORED RESEARCH OR DEVELOPMENT

Not applicable.

### BACKGROUND OF INVENTION

1. Field of the Invention

The present invention relates generally to vascular surgery, and more particularly to methods and graft devices for use in surgery, such as vascular anastomosis.

2. Background Art

With the advent of grafts, it has been possible for surgeons to replace various tissues, particularly, aorta and other major vessels, involved in diseases. However, such surgeries could be challenging and risky, leading to high morbidity and mortality.

Conventionally, graft implantation or replacement is performed with a suture. For example, after a surgeon performs a resection of an aortic tissue, usually a tube graft will be used to replace the section of the removed aorta. Surgeons will assess the aortic tissue and decide if pledges will be needed to perform the graft-aortic suture. Currently, surgeons use needles and stitches to perform this anastomosis. One of the challenges faced by surgeons is the occurrence of anastomotic bleeding in the walls of blood vessels, especially the back wall which is very difficult to access. To minimize this potential problem, some surgeons will perform the anastomosis using two layers, while others will perform this procedure using a piece of felt on both the inside and outside of the aorta. The challenging procedures often require significant heart lung bypass time, which will result in complications and/or slow recovery.

The costs involved in the care of patient with aortic surgery are significant. Bleeding that is difficult to control and prolonged operation time may contribute to morbidity and mortality. If the time spent doing the anastomosis in circulatory arrest is more than 30 minutes, it will increase the incidence of brain ischemia and stroke, resulting in significantly increased morbidity and mortality. Therefore, it has always been a goal of surgeons to reduce the operation time, morbidity, and mortality in aortic surgery, while performing a more reliable and efficient anastomosis.

### SUMMARY OF INVENTION

An aspect of the invention relates to grafts for a surgical procedures. A graft in accordance with one embodiment of the invention includes a graft body; and a double-layered section attached (which may be attached to the graft body or built in as one piece graft) to the graft body, wherein the double-layered section is configured to sandwich a target to be joined with the graft.

Another aspect of the invention relates to methods for implanting a graft. A method in accordance with one embodiment of the invention includes providing the graft having a double-layered section attached to a graft body; placing a target in the double-layered section such that the double-layered section embraces the target to form a sandwich structure; and joining the graft and the target by securing the sandwich structure. A method of the invention may eliminate the need for additional pledges or foreign bodies (such as a felt) to reinforce the anastomosis.

Other aspects and advantages of the invention will be apparent from the following description and the appended claims.

### BRIEF DESCRIPTION OF DRAWINGS

FIG. 1 shows a schematic illustrating a prior art method of anastomosis.

FIG. 2 shows a schematic illustrating an anastomosis in accordance with one embodiment of the invention.

FIGS. 3A-3D show partial cross sectional views of various grafts in accordance with embodiments of the invention.

FIG. 4 shows a graft having a locking mechanism in accordance with one embodiment of the invention.

FIGS. 5A and 5B show tubular grafts that may be made from sheet-like, flat grafts in accordance with embodiments of the invention.

FIG. 6 shows a leak-resistant graft in accordance with one embodiment of the invention.

### DETAILED DESCRIPTION

Embodiments of the invention relate to surgical grafts that can facilitate surgical procedures of graft implantation or replacement, such as vascular anastomosis. A graft in accordance with embodiments of the invention includes a unique feature that can facilitate and improve its joining with a target tissue (e.g., a blood vessel) or another graft. In accordance with some embodiments of the invention, the unique feature may include a double-layer structure configured to embrace (sandwich) the tissue to be joined. Some embodiments of the invention may further include locking mechanisms to facilitate the surgical procedures, hence reducing operation time and risk of complications that might result from prolonged heart-lung bypass.

While embodiments of the invention may be used in various tissue graft operations, for clarity of description, the following description will use vascular anastomosis for illustration. Therefore, while the description may refer to specific tissues such as vessels, aorta, veins, atrium, etc., these specific references are not intended to limit the scope of the invention.

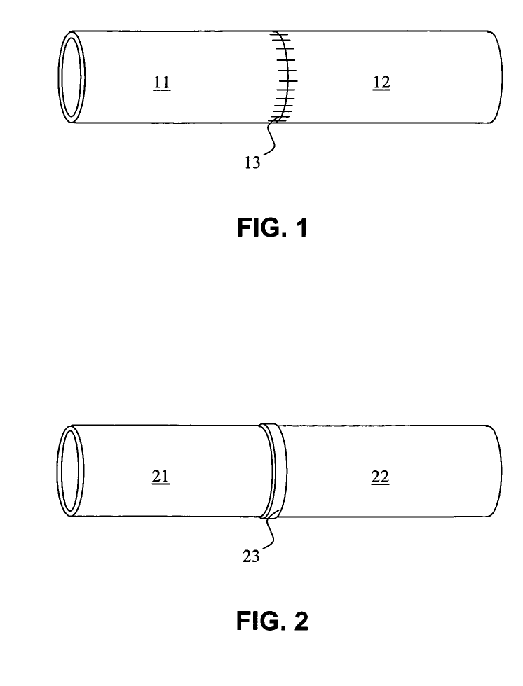

FIG. 1 shows a diagram illustrating a conventional surgical procedure in graft implantation or replacement. As shown, a graft 12 is joined with a blood vessel 11. The joining is typically accomplished with sutures 13 (or staples). However, suturing or stapling inevitably leaves gaps between stitches that are not securely annealed. These gaps may result in leaks. If blood leaks outside of the vessel, it may necessitate another operation or result in other complications.

FIG. 2 shows a schematic illustrating a joint made with a graft of the invention. As shown, a graft 22 in accordance with one embodiment of the invention is joined with a section of a blood vessel 21. The joint 23 comprises a double layered section of the graft 22 sandwiching one end of the blood vessel 21. The joint 23 may be secured with any means known in the art, including sutures, staples, or other mechanisms (e.g., locking mechanism described later). The double layered joint system (i.e., leak-resistant system) provides a stronger joint (due to the presence of two layers of graft materials). The joint thus formed is more resistant to leakage, i.e., it may provide a leak-resistant or leak-free joint. In this description, the double-layer joint system in accordance with embodiments of the invention will be referred to as the “sandwich joint” system or “leak-resistant” system.

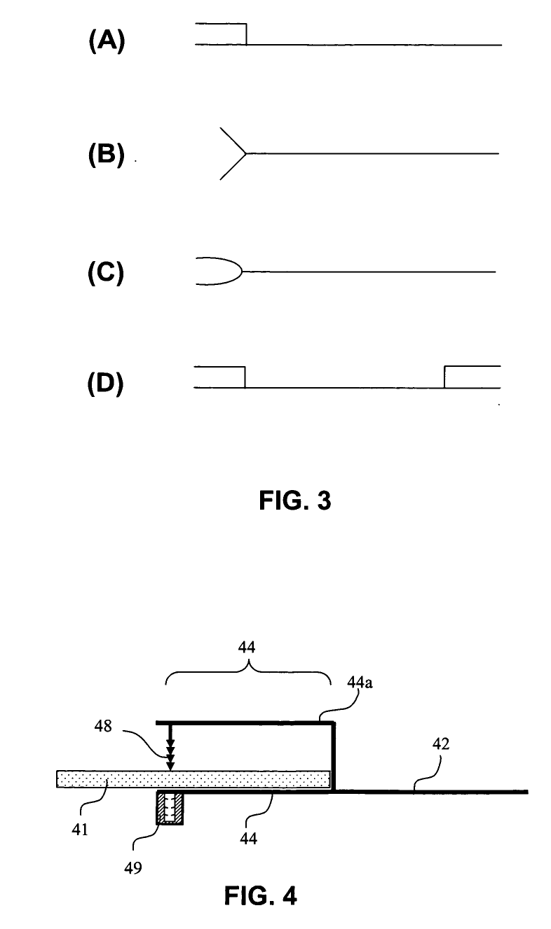

The double layered section on the graft 22 may be constructed in various configurations. FIGS. 3A-3C illustrate some suitable configurations in side way cross sectional views of grafts in accordance with embodiments of the invention. One of ordinary skill in the art would appreciate that these are examples only, and other modifications are possible without departing from the scope of the invention. Furthermore, one of ordinary skill in the art would appreciate that the sandwich joint mechanisms in accordance with embodiments of the invention may be used on both ends of a tubular grafts (FIG. 3D) or on other parts of other grafts that need to join with another tissue or another graft. In addition, a sandwich joint mechanism may be mounted on a flat structure (such as a pericardial graft), a round structure, or the like.

A tubular graft having the sandwich joint (leak-resistant) mechanisms of the invention at both ends, such as that shown in FIG. 3D, is particularly useful in “elephant trunk” operations. Elephant trunk operation (or elephant trunk technique) is a two-stage technique. In the first stage, the ascending aorta and aortic arch were replaced, leaving a segment of distal tubular graft in the descending thoracic aorta. In the second stage, the distal aorta is repaired beyond the subclavian artery. A tubular graft having leak-resistant systems at both ends will permit an aortic surgery to be performed in segments in different steps.

The double layer sections for forming a joint of the invention may have an inner layer that is a continuous layer such that it can prevent fluid from leaking to the outside. However, the outer layer need not be a complete, continuous layer. The outer layer may be in sections or have holes. In some embodiments, the outside layer may include one or more holes that allow a stay suture to be passed through. These holes may also be used to bring the tissue inside the pocket provided by the double layer structure.

As noted above, a joint in accordance with embodiments of the invention (shown as 23 in FIG. 2) may be secured with any means known in the medical technology, including sutures and staples. In addition, a graft in accordance with embodiments of the invention may include locking mechanisms to facilitate the joining procedures, hence to reduce operation time.

FIG. 4 illustrates a locking mechanism in accordance with one embodiment of the invention. As shown, a graft 42 in accordance with one embodiment of the invention includes a locking mechanism that comprises arrows 48 and corresponding recipient boxes 49, which can receive and lock the arrows. A plurality of arrows 48 and recipient boxes 49 may be placed around the circumference of a tubular graft, for example, or in other configurations suitable for the particular grafts. One of ordinary skill in the art would appreciate that the particular arrangement (configuration) of the arrows and recipient boxes on a graft is not important and should not limit the scope of the invention. After a tissue 41 (or another graft) is received in the double layered section 44 and sandwiched by the double layers 44a, 44b, the arrows 48 are pushed through the tissue 41 and locked with the recipient boxes 49 to securely join the tissue 41 and the graft 42. Note that several arrow heads are shown in series in each arrow 48 in FIG. 4. This is for illustration only and not intended to limit the scope of the invention. Instead, in accordance with embodiments of the invention, each arrow 48 may have one or more arrow heads.

The length of the arrows may depend on the target tissues that is to be joined with the grafts. In accordance with some embodiments of the invention, the length of the arrows may be optimized according to the thickness of the aorta or vessel walls to ensure proper hemostasis

One of ordinary skill in the art would appreciate that the arrow-recipient box shown in FIG. 4 is an example of a locking mechanism and other variations are possible without departing from the scope of the invention. For example, a locking mechanism may comprise a button that snaps into a complementary receiving piece (similar to snap buttons used in clothing) or any other locking mechanism. One of ordinary skill in the art would appreciate that while FIG. 4 shows locking mechanisms used with a graft having that particular configuration (as that shown in FIG. 3A), the locking mechanism may be used with a graft having other configurations (such as those shown in FIGS. 3B, 3C, 3D, or the like).

The graft-joining mechanisms in accordance with embodiments of the invention, i.e., using double layers to sandwich a tissue or another graft (the sandwich joint systems), provide stronger and more even joints. In addition, there is no gap between the layers making it less likely to leak. As shown in FIG. 6, a graft 62 in accordance with one embodiment of the invention comprise a double-layered section that includes a upper layer 63 and a lower layer 64. The upper layer 63 and lower layer 64 meet at a seam 65 to form continuous (i.e., no gap) fold along the entire length. While the example shown in FIG. 6 is a flat graft, the same feature is not limited to a flat graft. With a tubular graft, the seam 65 will be circular. The target (a tissue or another graft) to be sandwiched between the upper layer 63 and the lower layer 64 may be brought to abut the seam 65 such that a continuous seal is formed along the entire length. Alternatively, the complete contact between the target (not shown) and the upper layer 63 and/or the lower layer 64 may provide the sealing mechanism to prevent leakage. As a result, these joints are more leak-resistant or leak-proof.

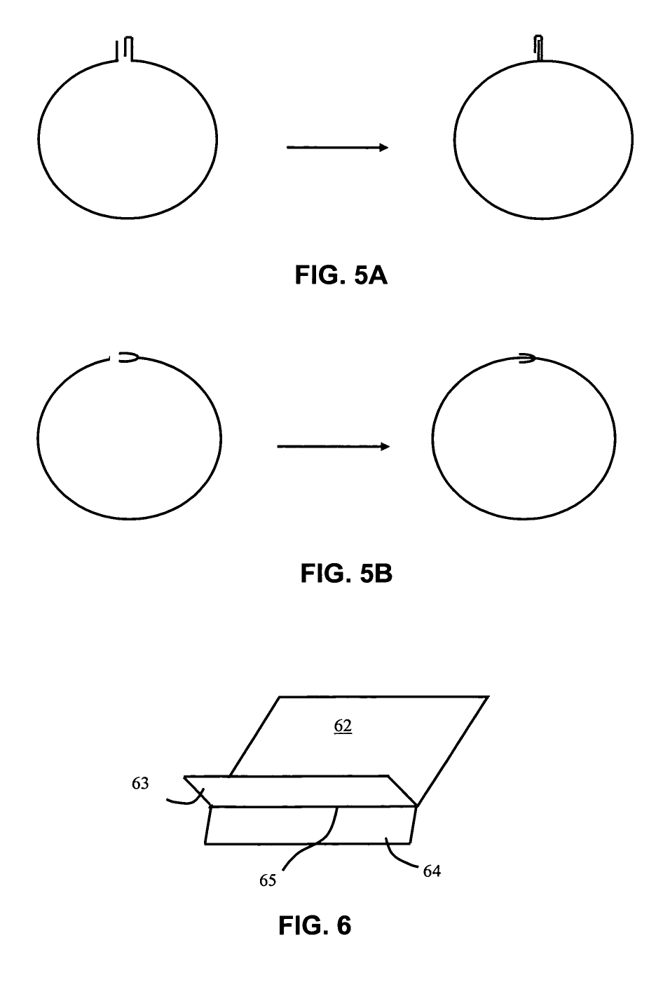

The ability to make leak-resistant or leak-proof joints makes it possible to join pieces of grafts or to construct surgical graft devices on the spot or during operations. FIGS. 5A and 5B illustrate cross-sectional views of tubular grafts that may be made from a sheet-like graft (a flat structure) as needed. The tubular grafts as shown in FIGS. 5A and 5B may be secured using sutures, staples, or any other locking/joining mechanisms (such as that shown in FIG. 4). The ability to make the graft devices as needed allows one to make a device with a diameter (or other dimensions) to fit the particular application. Note that while these examples use tubular grafts to illustrates embodiments of the invention, one of ordinary skill in the art would appreciate that the same approach may be used to make grafts of other shapes.

A graft in accordance with embodiments of the invention may be made of any graft material known in the art, including biological materials and/or synthetic materials (e.g., Dacron®, polytetrafluoroethylene (PTFE), PET (Polyethylene), polyglycolic acid, and polylatic acid). In addition, a graft of the invention may be manufactured using any suitable method known in the art. For example, U.S. Pat. No. 2,127,903 issued to Bowen discloses a bioabsorbable surgically implantable graft made of animal tissue and a binder; the graft is formed by wrapping strips of the treated animal tissue around a structural form. U.S. Pat. No. 4,909,979 issued to Possis discloses a method of shaping a human umbilical cord for use as a vascular graft. U.S. Pat. No. 6,190,590 issued to Randall et al. describe several methods for making PTFE graft devices. These patents are incorporated by reference in their entireties.

The double-layered section (leak-resistant or sandwich joint mechanism) and the remaining part of the graft (i.e., graft body) may be made as a unitary (integral) body. Alternatively, the double-layered section and the graft body may be separately manufactured and then joined or annealed together.

Advantages of embodiments of the invention may include one or more of the following. Embodiments of the present invention relate to systems of anastomosis of vascular structures, such as major vessels, with a significantly reduced risk of leaks. A system in accordance with embodiments of the invention provides a new concept in vascular surgery where a tube graft may be constructed as needed or during an operation, e.g., from flat to a round structure or from flat to a tubular structure. Embodiments of the present invention allow grafts to be custom built. In accordance with embodiments of the invention, a target tissue will be sandwiched between two layers of strong material. This system allows a more even distribution of forces among the suture line. This will considerably decrease the incidence of bleeding.

A system in accordance with embodiments of the invention can produce a stronger anastomosis, which will help decrease anastomotic bleeding. Embodiments of the invention allow quicker and safer anastomosis and more standardized anastomosis. A system of the invention may be applied from segments to individual areas. A system in accordance with the invention may be used to make various products: for example, a tube graft with the sandwich joint system (or leak-resistant system) for elephant trunk operation (which is replacement of the aorta with a tube graft leaving a distal limb of the graft for future second stage of the operation) and a pericardium patch with the leak-resistant device on the edge to be used for patching purposes or for patching of the atrium.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A graft for a surgical procedure, comprising:
a graft body; and
a double-layered section attached to the graft body, wherein the double-layered section is configured to sandwich a target to be joined with the graft.

2. The graft of claim 1, wherein the graft body and the double-layered section comprise a unitary piece.

3. The graft of claim 1, wherein the graft is made of a material selected from PTFE (Polytetrafluoroethylene), PET (Polyethylene), Dacron, polyglycolic acid, polylatic acid, pericardial patch, and a biologic material.

4. The graft of claim 1, further comprising a locking mechanism.

5. The graft of claim 4, wherein the locking mechanism comprising arrows and recipient boxes.

6. The graft of claim 1, wherein the target is a tissue.

7. The graft of claim 1, wherein the target is another graft.

8. The graft of claim 1, wherein the graft body is a tubular body and the double-layered section is attached to one end of the tubular body.

9. The graft of claim 8, further comprising a second double-layered section attached to the other end of the tubular body.

10. The graft of claim 1, wherein the graft body has a flat structure.

11. The graft of claim 10, wherein the double-layered section is attached to one edge of the flat structure.

12. A method for implanting a graft, comprising:
providing the graft having a double-layered section attached to a graft body;
placing a target in the double-layered section such that the double-layered section embraces the target to form a sandwich structure; and
joining the graft and the target by securing the sandwich structure.

13. The method of claim 12, wherein the securing the sandwich structure is by using a suture or a surgical staple.

14. The method of claim 12, wherein the securing the sandwich structure is by using a locking mechanism disposed in the double-layered section.

15. The method of claim 14, wherein the locking mechanism comprises arrows and recipient boxes.

16. The method of claim 12, wherein the target is a blood vessel.

17. The method of claim 12, wherein the target is another graft.
